(19) United States
(12) Patent Application Publication
(10) Pub. No.: US 2023/0125474 A1
(43) Pub. Date: Apr. 27, 2023

(54) SENSOR ELEMENT

(71) Applicant: NGK INSULATORS, LTD., Nagoya-si (JP)

(72) Inventors: Ryo ONISHI, Iwakura-shi (JP); Kaoru SHIBUTANI, Nagoya-shi (JP)

(21) Appl. No.: 17/950,631

(22) Filed: Sep. 22, 2022

(30) Foreign Application Priority Data
Oct. 22, 2021 (JP) 2021-173108

(51) Int. Cl. G01N 27/409; G01N 33/00; G01N 27/407

(52) U.S. Cl. CPC G01N 27/409; G01N 33/0037; G01N 27/4071; G01N 27/4076

(57) ABSTRACT

A sensor element for detecting a target gas to be measured in a measurement-object gas includes: an element body including an oxygen-ion-conductive solid electrolyte layer; and a protective layer covering at least a part of a surface of the element body. The protective layer includes a porous material that has a pore inside; and, in the pore in the protective layer, a ratio (Lt/Lf) of a pore length (Lt) in a thickness direction perpendicular to the surface of the element body to a pore length (Lf) in a surface direction perpendicular to the thickness direction is 0.6 to 0.9.

### CROSS-REFERENCE TO RELATED APPLICATION

The present application claims priority from Japanese application JP2021-173108, filed on Oct. 14, 2021, the contents of which is hereby incorporated by reference into this application.

### BACKGROUND OF THE INVENTION

### Technical Field of the Invention

The present invention relates to a sensor element in a gas sensor for detecting a target gas to be measured in a measurement-object gas.

### Background Art

A gas sensor is used for detection or measurement of concentration of an objective gas component (oxygen O₂, nitrogen oxide NOx, ammonia NH₃, hydrocarbon HC, carbon dioxide CO2, etc.) in a measurement-object gas, such as exhaust gas of automobile. For example, conventionally, the concentration of the objective gas component in exhaust gas of an automobile is measured, and an exhaust gas cleaning system mounted on the automobile is optimally controlled based on the measurement.

As such a gas sensor, a gas sensor equipped with a sensor element using an oxygen ion conductive solid electrolyte such as zirconia (ZrO₂) is known. It is also known that a porous protective layer is formed on a surface of the sensor element in such a gas sensor.

For example, JP 2016-065852 A discloses that a porous protective layer is formed by attaching a thermal spray powder such as alumina to the surface of a sensor element by plasma spraying.

### CITATION LIST

Patent Document
Patent Document 1: JP 2016-065852 A

### SUMMARY OF THE INVENTION

### Problems to be Solved by the Invention

In a gas sensor having a sensor element using a solid electrolyte, the sensor element has a high temperature (e.g., about 800° C.) during measurement of a target gas to be measured (during normal operation). Therefore, there is a problem that when water is splashed on the sensor element during normal operation of the gas sensor, only the surface of the sensor element having a high temperature is rapidly cooled due to the attachment of moisture so that cracking occurs in an internal structure of the sensor element due to the thermal shock.

Further, due to the tightening of automobile emission regulations, a gas sensor installed in an automobile is required to start to measure a target gas to be measured in exhaust gas just after starting of an automotive engine. However, just after engine starting, a larger amount of condensed water is present in exhaust pipes. Therefore, there is a higher risk that water is splashed on a sensor element having a high temperature.

Under such circumstances, the sensor element having a high temperature is required to further suppress the occurrence of cracking in its internal structure due to exposure to water (water splash). That is, there is an urgent need to improve the water resistance of the sensor element.

In light of this, it is an object of the present invention to provide a sensor element having high water resistance.

### Means for Solving the Problems

The present inventors have intensively studied, and as a result has found that the water resistance of the sensor element can be improved by forming a porous protective layer on at least a part of the surface of the sensor element and allowing pores of the protective layer to have a shape that spreads in a surface direction to be thin in a thickness direction of the protective layer (i.e., a so-called flat shape).

The present invention includes the following aspects.

(1) A sensor element for detecting a target gas to be measured in a measurement-object gas, the sensor element comprising:

an element body including an oxygen-ion-conductive solid electrolyte layer; and

a protective layer covering at least a part of a surface of the element body, wherein

the protective layer comprises a porous material that has a pore inside; and

in the pore in the protective layer, a ratio (Lt/Lf) of a pore length (Lt) in a thickness direction perpendicular to the surface of the element body to a pore length (Lf) in a surface direction perpendicular to said thickness direction is 0.6 to 0.9.

(2) The sensor element according to the above (1), wherein the protective layer has a thickness of 100 μm to 500 μm.
(3) The sensor element according to the above (1) or (2), wherein the protective layer has a porosity of 10% by volume to 40% by volume.
(4) The sensor element according to the above (1), wherein the protective layer comprises a surface layer, and an inner layer formed inside the surface layer; and

the inner layer has a higher porosity than the surface layer.

(5) The sensor element according to the above (4), wherein the inner layer in the protective layer has a thickness of 300 μm to 700 μm.
(6) The sensor element according to the above (4) or (5), wherein the surface layer in the protective layer has a thickness of 100 μm to 300 μm.
(7) The sensor element according to any one of the above (4) to (6), wherein the inner layer in the protective layer has a porosity of 40% by volume to 70% by volume.
(8) The sensor element according to any one of the above (1) to (7), wherein the sensor body comprises:

a base part in an elongated plate shape, including a plurality of oxygen-ion-conductive solid electrolyte layers stacked;

a measurement-object gas flow part formed from one end part in a longitudinal direction of the base part;

at least one inner electrode disposed on an inner surface of the measurement-object gas flow part; and

an outer electrode disposed in contact with the inner electrode via at least one layer of the plurality of oxygen-ion-conductive solid electrolyte layers.

(9) A production method of a sensor element according to any one of the above (1) to (8), the production method comprising the steps of:

applying a protective layer forming composition including a pore forming material, onto at least a part of a surface of the element body, to form a coating layer;

pressing the coating layer; and

degreasing the pressed coating layer to obtain a protective layer comprising a porous material.

### Advantageous Effect of the Invention

According to the present invention, it is possible to provide a sensor element having high water resistance.

### BRIEF DESCRIPTION OF THE DRAWINGS

FIG. 1 is a perspective view, showing one example of a general configuration of a sensor element 101.

FIG. 2 is a vertical sectional schematic view in the longitudinal direction, showing one example of a general configuration of a gas sensor 100 including the sensor element 101. FIG. 2 includes a sectional schematic view of the sensor element 101 along a line II-II in FIG. 1.

FIG. 3(A) is a schematic sectional view along a line III-III in FIG. 1. FIG. 3(A) is a schematic vertical sectional view orthogonal to the longitudinal direction of the sensor element 101. FIG. 3(B) is a schematic enlarged sectional view of a portion 3B of the porous protective layer 91a shown in FIG. 3(A), that is, a schematic view simply showing the shape of pores in the XZ section of the porous protective layer 91a by way of example.

FIG. 4(A) is a schematic diagram simply showing one example of the shape of pore precursors H in the section of the porous protective layer 91a after application. FIG. 4(B) is a schematic diagram simply showing one example of the shape of the pore precursors H in the section of the porous protective layer 91a after pressing.

### MODES FOR CARRYING OUT OF THE INVENTION

A sensor element of the present invention includes:

an element body including an oxygen-ion-conductive solid electrolyte layer; and

a protective layer covering at least a part of a surface of the element body, wherein

the protective layer comprises a porous material that has a pore inside; and

in the pore in the protective layer, a ratio (Lt/Lf) of a pore length (Lt) in a thickness direction perpendicular to the surface of the element body to a pore length (Lf) in a surface direction perpendicular to said thickness direction is 0.6 to 0.9.

Hereinafter, an example of an embodiment of a gas sensor having the sensor element of the present invention will be described in detail.

### [General Configuration of Gas Sensor]

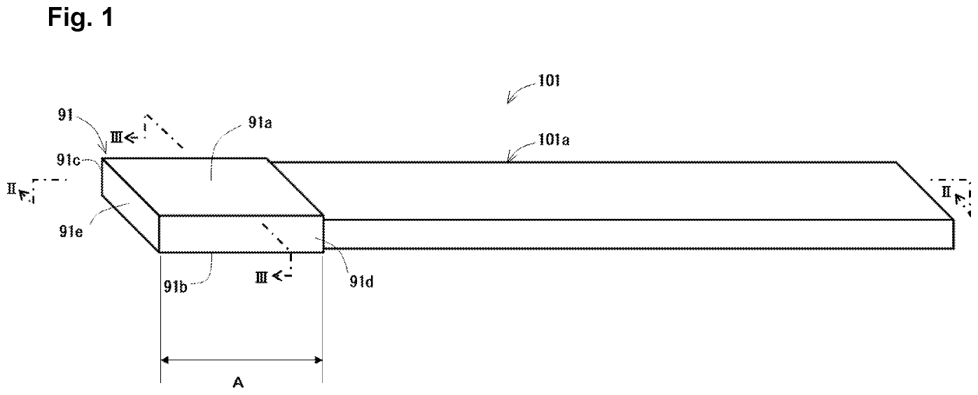

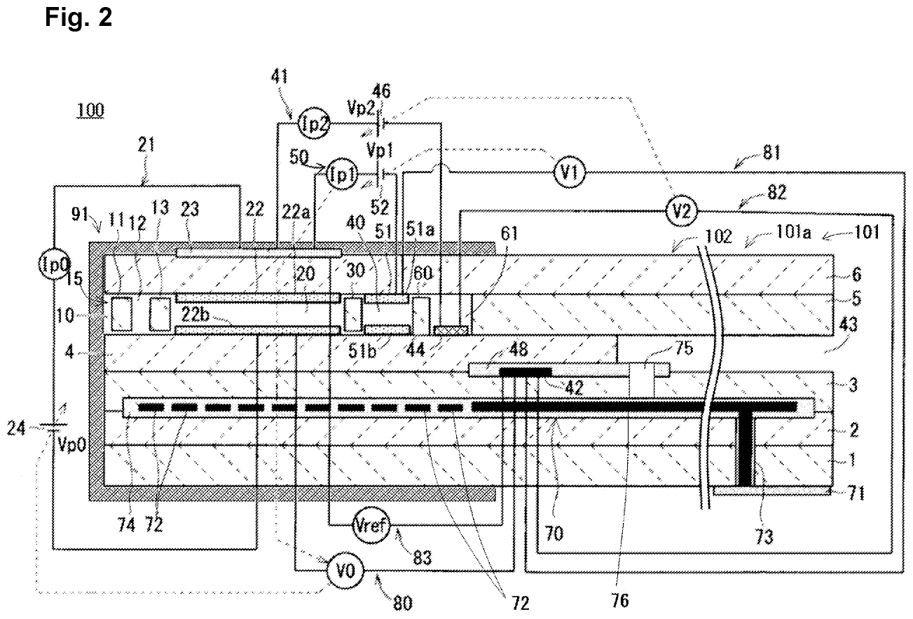

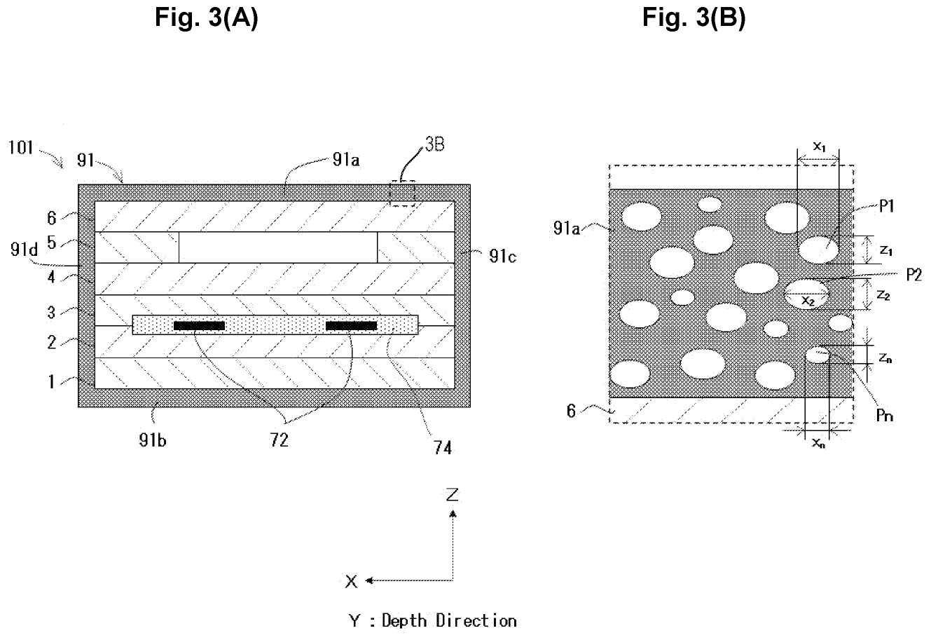

The gas sensor having sensor element of the present invention will now be described with reference to the drawings. FIG. 1 is a perspective view, showing one example of a general configuration of a sensor element 101. FIG. 2 is a vertical sectional schematic view in the longitudinal direction, showing one example of a general configuration of a gas sensor 100 including the sensor element 101. In FIG. 2, the sectional schematic view of the sensor element 101 is a sectional schematic view along a line II-II in FIG. 1. FIG. 3(A) is a schematic sectional view along a line III-III in FIG. 1. Hereinafter, based on FIG. 2, the upper side and the lower side in FIG. 2 are respectively defined as top and bottom, and the left side and the right side in FIG. 2 are respectively defined as a front end side and a rear end side. And, based on FIG. 3(A), the left side and the right side in FIG. 3(A) are respectively defined as a left side and a right side.

In FIG. 2, the gas sensor 100 represents one example of a limiting current type NOx sensor that detects NOx in a measurement-object gas by the sensor element 101, and measures the concentration of NOx.

The sensor element 101 includes a porous protective layer 91 that will be described later in detail. The porous protective layer 91 corresponds to the protective layer according to the present invention. The part of the sensor element 101 excluding the porous protective layer 91 is hereinafter referred to as an element body 101a.

In the sensor element 101 of this embodiment, an inner main pump electrode 22, an auxiliary pump electrode 51, and a measurement electrode 44 are provided as inner electrodes. As an outer electrode, an outer pump electrode 23 is provided.

The sensor element 101 is an element in an elongated plate shape, including a base part 102 having such a structure that a plurality of oxygen-ion-conductive solid electrolyte layers are layered. The elongated plate shape also called a long plate shape or a belt shape. The base part 102 has such a structure that six layers, namely, a first substrate layer 1, a second substrate layer 2, a third substrate layer 3, a first solid electrolyte layer 4, a spacer layer 5, and a second solid electrolyte layer 6, are layered in this order from the bottom side, as viewed in the drawing. Each of the six layers is formed of an oxygen-ion-conductive solid electrolyte layer containing, for example, zirconia (ZrO₂). The solid electrolyte forming these six layers is dense and gastight. These six layers all may have the same thickness, or the thickness may vary among the layers. The layers are adhered to each other with an adhesive layer of a solid electrolyte interposed therebetween, and the base part 102 includes the adhesive layer. While a layer configuration composed of the six layers is illustrated in FIG. 2, the layer configuration in the present invention is not limited to this, and any number of layers and any layer configuration are possible.

The sensor element 101 is manufactured, for example, by stacking ceramic green sheets corresponding to the individual layers after conducting predetermined processing, printing of circuit pattern and the like, and then firing the stacked ceramic green sheets so that they are combined together.

A gas inlet 10 is formed between the lower surface of the second solid electrolyte layer 6 and the upper surface of the first solid electrolyte layer 4 in one end part in the longitudinal direction (hereinafter, referred to as a front end part) of the sensor element 101. A measurement-object gas flow part 15 is formed in such a form that a first diffusion-rate limiting part 11, a buffer space 12, a second diffusion-rate limiting part 13, a first internal cavity 20, a third diffusion-rate limiting part 30, a second internal cavity 40, a fourth diffusion-rate limiting part 60, and a third internal cavity 61 communicate in this order in the longitudinal direction from the gas inlet 10.

The gas inlet 10, the buffer space 12, the first internal cavity 20, the second internal cavity 40, and the third internal cavity 61 constitute internal spaces of the sensor element 101. Each of the internal spaces is provided in such a manner that a portion of the spacer layer 5 is hollowed out, and the top of each of the internal spaces is defined by the lower surface of the second solid electrolyte layer 6, the bottom of each of the internal spaces is defined by the upper surface of the first solid electrolyte layer 4, and the lateral surface of each of the internal spaces is defined by the lateral surface of the spacer layer 5.

Each of the first diffusion-rate limiting part 11, the second diffusion-rate limiting part 13, and the third diffusion-rate limiting part 30 is provided as two laterally elongated slits (having the longitudinal direction of the openings in the direction perpendicular to the figure in FIG. 2). Each of the first diffusion-rate limiting part 11, the second diffusion-rate limiting part 13, and the third diffusion-rate limiting part 30 may be in such a form that a desired diffusion resistance is created, but the form is not limited to the slits.

The fourth diffusion-rate limiting part 60 is provided as a single laterally elongated slit (having the longitudinal direction of the opening in the direction perpendicular to the figure in FIG. 2) between the spacer layer 5 and the second solid electrolyte layer 6. The fourth diffusion-rate limiting part 60 may be in such a form that a desired diffusion resistance is created, but the form is not limited to the slit.

Also, at a position farther from the front end than the measurement-object gas flow part 15, a reference gas introduction space 43 is disposed between the upper surface of the third substrate layer 3 and the lower surface of the spacer layer 5 at a position where the reference gas introduction space 43 is laterally defined by the lateral surface of the first solid electrolyte layer 4. The reference gas introduction space 43 has an opening in the other end part (hereinafter, referred to as a rear end part) of the sensor element 101. As a reference gas for NOx concentration measurement, for example, air is introduced into the reference gas introduction space 43.

An air introduction layer 48 is a layer formed of porous alumina, and is so configured that a reference gas is introduced into the air introduction layer 48 via the reference gas introduction space 43. The air introduction layer 48 is formed to cover a reference electrode 42.

The reference electrode 42 is an electrode sandwiched between the upper surface of the third substrate layer 3 and the first solid electrolyte layer 4, and as described above, the air introduction layer 48 leading to the reference gas introduction space 43 is disposed around the reference electrode 42. That is, the reference electrode 42 is disposed to be in contact with a reference gas via the air introduction layer 48 which is a porous material, and the reference gas introduction space 43. As will be described later, the reference electrode 42 can be used to measure the oxygen concentration (oxygen partial pressure) in the first internal cavity 20, the second internal cavity 40, and the third internal cavity 61.

In the measurement-object gas flow part 15, the gas inlet 10 is open to the external space, and the measurement-object gas is taken into the sensor element 101 from the external space through the gas inlet 10.

In the present embodiment, the measurement-object gas flow part 15 is in such a form that the measurement-object gas is introduced through the gas inlet 10 that is open on the front end surface of the sensor element 101, however, the present invention is not limited to this form. For example, the measurement-object gas flow part 15 need not have a recess of the gas inlet 10. In this case, the first diffusion-rate limiting part 11 substantially serves as a gas inlet.

For example, the measurement-object gas flow part 15 may have an opening that communicates with the buffer space 12 or a position near the buffer space 12 of the first internal cavity 20, on a lateral surface along the longitudinal direction of the base part 102. In this case, the measurement-object gas is introduced from the lateral surface along the longitudinal direction of the base part 102 through the opening.

Further, for example, the measurement-object gas flow part 15 may be so configured that the measurement-object gas is introduced through a porous body.

The first diffusion-rate limiting part 11 creates a predetermined diffusion resistance to the measurement-object gas taken through the gas inlet 10.

The buffer space 12 is provided to guide the measurement-object gas introduced from the first diffusion-rate limiting part 11 to the second diffusion-rate limiting part 13.

The second diffusion-rate limiting part 13 creates a predetermined diffusion resistance to the measurement-object gas introduced into the first internal cavity 20 from the buffer space 12.

It suffices that the amount of the measurement-object gas to be introduced into the first internal cavity 20 finally falls within a predetermined range. That is, it suffices that a predetermined diffusion resistance is created in a whole from the front end part of the sensor element 101 to the second diffusion-rate limiting part 13. For example, the first diffusion-rate limiting part 11 may directly communicate with the first internal cavity 20, or the buffer space 12 and the second diffusion-rate limiting part 13 may be absent.

The buffer space 12 is provided to mitigate the influence of pressure fluctuation on the detected value when the pressure of the measurement-object gas fluctuates.

When the measurement-object gas is introduced from outside the sensor element 101 into the first internal cavity 20, the measurement-object gas, which is rapidly taken through the gas inlet 10 into the sensor element 101 due to pressure fluctuation of the measurement-object gas in the external space (pulsations in exhaust pressure if the measurement-object gas is automotive exhaust gas), is not directly introduced into the first internal cavity 20. Rather, the measurement-object gas is introduced into the first internal cavity 20 after the pressure fluctuation of the measurement-object gas is eliminated through the first diffusion-rate limiting part 11, the buffer space 12, and the second diffusion-rate limiting part 13. Thus, the pressure fluctuation of the measurement-object gas introduced into the first internal cavity 20 becomes almost negligible.

The first internal cavity 20 is provided as a space for adjusting the oxygen partial pressure in the measurement-object gas introduced through the second diffusion-rate limiting part 13. The oxygen partial pressure is adjusted by operation of a main pump cell 21.

The main pump cell 21 is an electrochemical pump cell including the inner main pump electrode 22 as an inner electrode disposed on the inner surface of the measurement-object gas flow part 15, and the outer pump electrode 23 as an outer electrode disposed in contact with the inner main pump electrode 22 via a solid electrolyte (in FIG. 2, via the second solid electrolyte layer 6).

That is, the main pump cell 21 is an electrochemical pump cell composed of the inner main pump electrode 22 having a ceiling electrode portion 22a disposed over substantially the entire surface of the lower surface of the second solid electrolyte layer 6 that faces the first internal cavity 20, the outer pump electrode 23 disposed on a region of the upper surface of the second solid electrolyte layer 6 that corresponds to the ceiling electrode portion 22a so as to be exposed to the external space, and the second solid electrolyte layer 6 sandwiched between the inner main pump electrode 22 and the outer pump electrode 23.

The inner main pump electrode 22 is formed to span the upper and lower solid electrolyte layers (the second solid electrolyte layer 6 and the first solid electrolyte layer 4) that define the first internal cavity 20 and the spacer layer 5 that defines the lateral wall. Specifically, the ceiling electrode portion 22a is formed on the lower surface of the second solid electrolyte layer 6 that defines the ceiling surface of the first internal cavity 20, and a bottom electrode portion 22b is formed on the upper surface of the first solid electrolyte layer 4 that defines the bottom surface of the first internal cavity 20. Also, lateral electrode portions (not shown) are formed on the lateral wall surfaces (inner surface) of the spacer layer 5 that form both lateral wall parts of the first internal cavity 20 so as to connect the ceiling electrode portion 22a and the bottom electrode portion 22b. Thus, the inner main pump electrode 22 is provided as a tunnel-like structure in the area where the lateral electrode portions are disposed.

The inner main pump electrode 22 and the outer pump electrode 23 are each formed as a porous cermet electrode (e.g., a cermet electrode of Pt containing 1% Au and ZrO₂). It is to be noted that the inner main pump electrode 22 to be in contact with the measurement-object gas is formed using a material having a weakened ability to reduce a NOx component in the measurement-object gas.

In the main pump cell 21, a desired pump voltage Vp0 is applied between the inner main pump electrode 22 and the outer pump electrode 23 by a variable power supply 24 to flow a pump current Ip0 between the inner main pump electrode 22 and the outer pump electrode 23 in either a positive or negative direction, and thus it is possible to pump out oxygen in the first internal cavity 20 to the external space or pump oxygen into the first internal cavity 20 from the external space.

To detect the oxygen concentration (oxygen partial pressure) in the atmosphere in the first internal cavity 20, the inner main pump electrode 22, the second solid electrolyte layer 6, the spacer layer 5, the first solid electrolyte layer 4, the third substrate layer 3, and the reference electrode 42 form an electrochemical sensor cell, namely, an oxygen-partial-pressure detection sensor cell 80 for main pump control.

The oxygen concentration (oxygen partial pressure) in the first internal cavity 20 can be detected from an electromotive force V0 measured in the oxygen-partial-pressure detection sensor cell 80 for main pump control. In addition, the pump current Ip0 is controlled by performing feedback control of the pump voltage Vp0 in the variable power supply 24 so that the electromotive force V0 is constant. Thus, the oxygen concentration in the first internal cavity 20 can be maintained at a predetermined constant value.

The third diffusion-rate limiting part 30 creates a predetermined diffusion resistance to the measurement-object gas whose oxygen concentration (oxygen partial pressure) has been controlled in the first internal cavity 20 by the operation of the main pump cell 21, and guides the measurement-object gas into the second internal cavity 40.

The second internal cavity 40 is provided as a space for adjusting the oxygen partial pressure in the measurement-object gas introduced through the third diffusion-rate limiting part 30 more accurately. The oxygen partial pressure is adjusted by operation of an auxiliary pump cell 50. The sensor element 101 may be configured without the second internal cavity 40 and the auxiliary pump cell 50. From the viewpoint of adjusting accuracy of oxygen partial pressure, it is more preferred that the second internal cavity 40 and the auxiliary pump cell 50 be provided.

After the oxygen concentration (oxygen partial pressure) in the measurement-object gas is adjusted in advance in the first internal cavity 20, the measurement-object gas is introduced through the third diffusion-rate limiting part 30, and is further subjected to adjustment of the oxygen partial pressure by the auxiliary pump cell 50 in the second internal cavity 40. Thus, the oxygen concentration in the second internal cavity 40 can be kept constant with high accuracy, and the NOx concentration can be measured with high accuracy in the gas sensor 100.

The auxiliary pump cell 50 is an electrochemical pump cell including the auxiliary pump electrode 51 as an inner electrode disposed at a position farther from the front end portion in the longitudinal direction of the base part 102 than the inner main pump electrode 22 on the inner surface of the measurement-object gas flow part 15, and the outer pump electrode 23 as an outer electrode disposed in contact with the auxiliary pump electrode 51 via a solid electrolyte (in FIG. 2, via the second solid electrolyte layer 6).

That is, the auxiliary pump cell 50 is an auxiliary electrochemical pump cell composed of the auxiliary pump electrode 51 having a ceiling electrode portion 51a disposed on substantially the entire surface of the lower surface of the second solid electrolyte layer 6 facing with the second internal cavity 40, the outer pump electrode 23 (the outer electrode is not limited to the outer pump electrode 23, but may be any suitable electrode outside the sensor element 101), and the second solid electrolyte layer 6.

This auxiliary pump electrode 51 is disposed in the second internal cavity 40 in a tunnel-like structure similar to the inner main pump electrode 22 disposed in the first internal cavity 20 described previously. Specifically, in the tunnel-like structure, the ceiling electrode portion 51a is formed on the lower surface of the second solid electrolyte layer 6 that defines the ceiling surface of the second internal cavity 40, a bottom electrode portion 51b is formed on the upper surface of the first solid electrolyte layer 4 that defines the bottom surface of the second internal cavity 40, and lateral electrode portions (not shown) connecting the ceiling electrode portion 51a and the bottom electrode portion 51b are formed on the wall surfaces of the spacer layer 5 that define the lateral walls of the second internal cavity 40.

It is to be noted that the auxiliary pump electrode 51 is formed using a material having a weakened ability to reduce a NOx component in the measurement-object gas, as with the case of the inner main pump electrode 22.

In the auxiliary pump cell 50, by applying a desired voltage Vp1 between the auxiliary pump electrode 51 and the outer pump electrode 23, it is possible to pump out oxygen in the atmosphere in the second internal cavity 40 to the external space, or pump the oxygen into the second internal cavity 40 from the external space.

To control the oxygen partial pressure in the atmosphere in the second internal cavity 40, the auxiliary pump electrode 51, the reference electrode 42, the second solid electrolyte layer 6, the spacer layer 5, the first solid electrolyte layer 4, and the third substrate layer 3 constitute an electrochemical sensor cell, namely, an oxygen-partial-pressure detection sensor cell 81 for auxiliary pump control.

The auxiliary pump cell 50 performs pumping with a variable power supply 52 whose voltage is controlled on the basis of an electromotive force V1 detected by the oxygen-partial-pressure detection sensor cell 81 for auxiliary pump control. Thus, the oxygen partial pressure in the atmosphere in the second internal cavity 40 is controlled to such a low partial pressure that does not substantially affect measurement of NOx.

In addition, a pump current Ip1 is used for control of the electromotive force of the oxygen-partial-pressure detection sensor cell 80 for main pump control. Specifically, the pump current Ip1 is input to the oxygen-partial-pressure detection sensor cell 80 for main pump control as a control signal to control the electromotive force V0, and thus the gradient of the oxygen partial pressure in the measurement-object gas introduced into the second internal cavity 40 from the third diffusion-rate limiting part 30 is controlled to remain constant. In using as a NOx sensor, the oxygen concentration in the second internal cavity 40 is kept at a constant value of about 0.001 ppm by the actions of the main pump cell 21 and the auxiliary pump cell 50.

The fourth diffusion-rate limiting part 60 creates a predetermined diffusion resistance to the measurement-object gas whose oxygen concentration (oxygen partial pressure) has been controlled to further low in the second internal cavity 40 by the operation of the auxiliary pump cell 50, and guides the measurement-object gas into the third internal cavity 61.

The third internal cavity 61 is provided as a space for measuring nitrogen oxide (NOx) concentration in the measurement-object gas introduced through the fourth diffusion-rate limiting part 60. By the operation of a measurement pump cell 41, NOx concentration is measured.

The measurement pump cell 41 is an electrochemical pump cell including a measurement electrode 44 as an inner electrode disposed at a position farther from the front end portion in the longitudinal direction of the base part 102 than the auxiliary pump electrode 51 on the inner surface of the measurement-object gas flow part 15, and the outer pump electrode 23 as an outer electrode disposed in contact with the measurement electrode 44 via a solid electrolyte (in FIG. 2, via the second solid electrolyte layer 6, the spacer layer 5, and the first solid electrolyte layer 4).

That is, the measurement pump cell 41 measures NOx concentration in the measurement-object gas in the third internal cavity 61. The measurement pump cell 41 is an electrochemical pump cell composed of the measurement electrode 44 disposed on the upper surface of the first solid electrolyte layer 4 facing with the third internal cavity 61, the outer pump electrode 23 (the outer electrode is not limited to the outer pump electrode 23, but may be any suitable electrode outside the sensor element 101), the second solid electrolyte layer 6, the spacer layer 5, and the first solid electrolyte layer 4.

The measurement electrode 44 is a porous cermet electrode. The measurement electrode 44 functions also as a NOx reduction catalyst that reduces NOx present in the atmosphere in the third internal cavity 61.

In the measurement pump cell 41, oxygen generated by decomposition of nitrogen oxide in the atmosphere around the measurement electrode 44 is pumped out, and the amount of generated oxygen can be detected as a pump current Ip2.

To detect the oxygen partial pressure around the measurement electrode 44, the second solid electrolyte layer 6, the spacer layer 5, the first solid electrolyte layer 4, the third substrate layer 3, the measurement electrode 44, and the reference electrode 42 constitute an electrochemical sensor cell, namely an oxygen-partial-pressure detection sensor cell 82 for measurement pump control. A variable power supply 46 is controlled on the basis of an electromotive force V2 detected by the oxygen-partial-pressure detection sensor cell 82 for measurement pump control.

The measurement-object gas introduced into the second internal cavity 40 reaches the measurement electrode 44 through the fourth diffusion-rate limiting part 60 under the condition that the oxygen partial pressure is controlled. Nitrogen oxide in the measurement-object gas around the measurement electrode 44 is reduced (2NO→N₂+O₂) to generate oxygen. The generated oxygen is to be pumped by the measurement pump cell 41, and at this time, a voltage Vp2 of the variable power supply 46 is controlled so that the electromotive force V2 detected by the oxygen-partial-pressure detection sensor cell 82 for measurement pump control is constant. Since the amount of oxygen generated around the measurement electrode 44 is proportional to the concentration of nitrogen oxide in the measurement-object gas, nitrogen oxide concentration in the measurement-object gas is calculated by using the pump current Ip2 in the measurement pump cell 41.

By configuring oxygen partial pressure detecting means by an electrochemical sensor cell composed of a combination of the measurement electrode 44, the first solid electrolyte layer 4, the third substrate layer 3 and the reference electrode 42, it is possible to detect an electromotive force in accordance with a difference between the amount of oxygen generated by reduction of NOx components in the atmosphere around the measurement electrode 44 and the amount of oxygen contained in the reference air, and hence it is possible to determine the concentration of NOx components in the measurement-object gas.

Also, the second solid electrolyte layer 6, the spacer layer 5, the first solid electrolyte layer 4, the third substrate layer 3, the outer pump electrode 23, and the reference electrode 42 constitute an electrochemical sensor cell 83, and it is possible to detect the oxygen partial pressure in the measurement-object gas outside the sensor by an electromotive force Vref obtained by the sensor cell 83.

In the gas sensor 100 having such a configuration, the main pump cell 21 and the auxiliary pump cell 50 are operated to supply a measurement-object gas whose oxygen partial pressure is usually kept at a low constant value (the value that does not substantially affect measurement of NOx) to the measurement pump cell 41. Therefore, NOx concentration in the measurement-object gas can be detected on the basis of the pump current Ip2 that flows as a result of pumping out of the oxygen generated by reduction of NOx by the measurement pump cell 41 and is almost in proportion to the concentration of NOx in the measurement-object gas.

In the present embodiment, the sensor element 101 is configured to have three internal cavities, namely, the first internal cavity 20, the second internal cavity 40 and the third internal cavity 61, and configured that the internal electrodes 22, 51, 44 are disposed in each of the internal cavities 20, 40, 61, respectively. However, the number of internal cavities and the arrangement configuration of internal cavities are not limited to this embodiment. The number of internal cavities may be one or two, or may be 4 or more.

The sensor element 101 further includes a heater part 70 that functions as a temperature regulator of heating and maintaining the temperature of the sensor element 101 so as to enhance the oxygen ion conductivity of the solid electrolyte. The heater part 70 includes a heater electrode 71, a heater 72, a heater lead 76, a through hole 73, a heater insulating layer 74, and a pressure relief vent 75.

The heater electrode 71 is an electrode formed in contact with the lower surface of the first substrate layer 1. The power can be supplied to the heater part 70 from the outside by connecting the heater electrode 71 with a heater power supply that is an external power supply.

The heater 72 is an electrical resistor sandwiched by the second substrate layer 2 and the third substrate layer 3 from top and bottom. The heater 72 is connected with the heater electrode 71 via a heater lead 76 that connects with the heater 72 and extends in the rear end side in the longitudinal direction of the sensor element 101, and the through hole 73. The heater 72 is externally powered through the heater electrode 71 to generate heat, and heats and maintains the temperature of the solid electrolyte forming the sensor element 101.

The heater 72 is embedded over the whole area from the first internal cavity 20 to the third internal cavity 61 so that the temperature of the entire sensor element 101 can be adjusted to such a temperature that activates the solid electrolyte. The temperature may be adjusted so that the main pump cell 21, the auxiliary pump cell 50, and the measurement pump cell 41 are operable. It is not necessary that the whole area is adjusted to the same temperature, but the sensor element 101 may have temperature distribution.

In the sensor element 101 of the present embodiment, the heater 72 is embedded in the base part 102, but this form is not limitative. The heater 72 may be disposed to heat the base part 102. That is, the heater 72 may heat the sensor element 101 to develop oxygen ion conductivity with which the main pump cell 21, the auxiliary pump cell 50, and the measurement pump cell 41 are operable. For example, the heater 72 may be embedded in the base part 102 as in the present embodiment. Alternatively, for example, the heater part 70 may be formed as a heater substrate that is separate from the base part 102, and may be disposed at a position adjacent to the base part 102.

The heater insulating layer 74 is formed of an insulator such as alumina on the upper and lower surfaces of the heater 72 and the heater lead 76. The heater insulating layer 74 is formed to ensure electrical insulation between the second substrate layer 2, and the heater 72 and the heater lead 76, and electrical insulation between the third substrate layer 3, and the heater 72 and the heater lead 76.

The pressure relief vent 75 extends through the third substrate layer 3 so that the heater insulating layer 74 and the reference gas introduction space 43 communicate with each other. The pressure relief vent 75 can mitigate an increase in internal pressure due to temperature rise in the heater insulating layer 74. The pressure relief vent 75 may be absent.

### (Protective Layer)

The sensor element 101 includes the element body 101a and the porous protective layer 91 covering a part of the element body 101a. In this embodiment, as shown in FIG. 1, the porous protective layer 91 includes porous protective layers 91a to 91e. The porous protective layer 91a entirely covers a part of the top surface of the element body 101a which extends for a distance A in the longitudinal direction from the front end of the element body 101a. The porous protective layer 91b entirely covers a part of the bottom surface of the element body 101a which extends for a distance A in the longitudinal direction from the front end of the element body 101a. The porous protective layer 91c entirely covers a part of the right surface of the element body 101a which extends for a distance A in the longitudinal direction from the front end of the element body 101a. The porous protective layer 91d entirely covers a part of the left surface of the element body 101a which extends for a distance A in the longitudinal direction from the front end of the element body 101a. The porous protective layer 91e entirely covers the front end surface of the element body 101a.

The porous protective layer 91e also covers the gas inlet 10. However, a measurement-object gas can reach the gas inlet 10 through the inside of the porous protective layer 91e because the porous protective layer 91e is a porous material. Therefore, a target gas to be measured can be detected and measured without problem.

The porous protective layer 91 plays a role of suppressing the occurrence of cracking in the internal structure of the element body 101a when, for example, water is splashed on the sensor element 101 having a high temperature during normal operation of the gas sensor. Water that has reached the sensor element 101 is not directly attached to the surface of the element body 101a but is attached to the porous protective layer 91. The surface of the porous protective layer 91 is rapidly cooled by the attached water, but thermal shock applied to the element body 101a is reduced by the heat insulating effect of the porous protective layer 91. This, as a result, makes it possible to suppress the occurrence of cracking in the internal structure of the element body 101a. That is, the water resistance of the sensor element 101 improves.

The porous protective layer 91a covers the outer pump electrode 23. The porous protective layer 91a also plays a role of suppressing the attachment of an oil component or the like contained in a measurement-object gas to the outer pump electrode 23 to prevent degradation of the outer pump electrode 23.

The porous protective layer 91 in this embodiment entirely covers a part of the element body 101a (91a, 91b, 91c, 91d, 91e) which includes its front end surface and extends for a distance A in the longitudinal direction of the element body 101a from the front end surface. The distance A should be determined to fall within a range of 0<distance A<entire longitudinal length of element body 101a on the basis of the area of the element body 101a to be exposed to a measurement-object gas in the gas sensor 100, the position of the outer pump electrode 23, or the like. The porous protective layers 91a to 91d may be different from each other in their lengths in the longitudinal direction of the element body 101a.

The porous protective layer 91 should be formed on at least one of the front end surface, top and bottom surfaces, and right and left surfaces of the element body 101a. For example, the porous protective layer 91 may be formed on only the top surface or may be formed on the two top and bottom surfaces.

The porous protective layer 91 comprises a porous material. Examples of a constituent material of the porous protective layer 91 include alumina, zirconia, spinel, cordierite, mullite, titania, and magnesium. Any one or two or more of them may be used. In this embodiment, the porous protective layer 91 comprises an alumina porous material.

In a pore present inside the porous protective layer 91, a ratio (Lt/Lf) of a pore length (Lt) in a thickness direction perpendicular to the surface of the element body 101a to a pore length (Lf) in a surface direction perpendicular to the thickness direction is 0.6 to 0.9. That is, pores present inside the porous protective layer 91 averagely have a shape that spreads in the surface direction to be thin in the thickness direction of the porous protective layer 91 (i.e., a so-called flat shape).

FIG. 3(A) is a schematic sectional view along a line III-III in FIG. 1, that is, a schematic vertical sectional view orthogonal to the longitudinal direction of the sensor element 101. In FIG. 3(A), the outer pump electrode 23 and the inner main pump electrode 22 are not shown. In the following description, the horizontal direction, the vertical direction, and the direction perpendicular to the paper surface of the drawing in FIG. 3(A) are referred to as an X axis direction, a Z axis direction, and a Y axis direction, respectively. The X axis direction is a direction perpendicular to the longitudinal direction of the sensor element 101 and parallel to the surfaces of the solid electrolyte layers 1 to 6 (i.e., the width direction of the sensor element 101). The Y axis direction corresponds to the longitudinal direction of the sensor element 101. The Z axis direction is a direction perpendicular to the longitudinal direction of the sensor element 101 and perpendicular to the surfaces of the solid electrolyte layers 1 to 6 (i.e., the thickness direction of the sensor element 101).

FIG. 3(B) is a schematic enlarged sectional view of the porous protective layer 91a shown in FIG. 3(A), that is, a schematic view simply showing the shape of pores in the XZ section of the porous protective layer 91a by way of example. The Z axis direction corresponds to the thickness direction of the porous protective layer 91a. The shape of the pores is not limited to such an almost elliptical shape as shown by way of example in FIG. 3(B), and the pores may have various shapes. The size, number, and distribution state of the pores are not limited to those shown by way of example in FIG. 3(B). Although FIG. 3(B) shows the porous protective layer 91a by way of example, the same goes for the porous protective layers 91b to 91e.

Most of the pores inside the porous protective layer 91 each have a communicating portion not shown in FIG. 3(B) to form a structure such that the communicating portion is interposed between the pore and one or two or more pores adjacent to it or between the pore and one or two or more pores close to it. It is to be noted that the communicating portion constitutes a part of the pore. The pores close to a surface of the porous protective layer 91 are often open to the surface. The pores close to an interface with the element body 101a are also often open to the interface.

In the present invention, the pore length (Lt) in a thickness direction perpendicular to the surface of the element body 101a corresponds to the average of pore lengths in the thickness direction of all the pores present inside the porous protective layer 91. The pore length (Lf) in a surface direction perpendicular to the thickness direction corresponds to the average of pore lengths in the surface direction of all the pores present inside the porous protective layer 91. In the porous protective layer 91a, the surface direction corresponds to the X axis direction (i.e., the width direction of the sensor element 101). The surface direction may correspond to the Y axis direction (i.e., the longitudinal direction of the sensor element 101). The reason for this is as follows. The pores have various shapes, and therefore the pore length in the X axis direction and the pore length in the Y axis direction of each of the pores are usually different. However, when a comparison is made between their averages, they are considered to be equivalent.

The ratio (Lt/Lf) conceptually corresponds to the following value. For example, the section shown in FIG. 3(B) is given as an example, which has n pores P1, P2, . . . Pn. The lengths in the thickness direction of the pores P1, P2 . . . Pn are respectively defined as z1, z2, . . . zn, and the lengths in the surface direction of the pores P1, P2 . . . Pn are respectively defined as x1, x2, . . . xn. In this case,

the pore length in the thickness direction is expressed as (Lt)=(z1+z2+ . . . +zn)/n]; and

the pore length in the surface direction is expressed as (Lf)=[(x1+x2+ . . . +xn)/n].

The ratio (Lt/Lf) is a ratio of the pore length (Lt) in the thickness direction to the pore length (Lf) in the surface direction.

In the actual section of the porous protective layer 91, there are various shapes of pores including the communicating portions. Specifically, the ratio (Lt/Lf) in the present invention is determined in the following manner. The ratio (Lt/Lf) is determined in the following procedure by the image analysis of CT (Computed Tomography) image of the porous protective layer 91.

1) The microstructure of the porous protective layer 91 of the sensor element 101 is imaged by CT.
2) The image of XZ section of the porous protective layer 91a is obtained at any position. The horizontal direction and the vertical direction of the sectional image are respectively defined as an X axis direction and a Z axis direction. The number of pixels in the sectional image is 600 pixels (width)×80 pixels (height), and 1 pixel is 1.5 micrometers square.
3) The obtained sectional image is binarized using “Otsu's method” (also referred to as discriminant analysis method). In the binarized sectional image, the constituent material of the porous protective layer 91 (in this embodiment, alumina) is shown in white and pores are shown in black.
4) In the rightmost vertical (Z axis direction) line of 1 pixel width in the sectional image, the number of black pixels that are continuous vertically (in the Z axis direction) in each series of one or more black pixels separated by white pixels is counted, and the average of the numbers is calculated. Also in each of all the second and subsequent vertical (Z axis direction) lines of 1 pixel width from the right in the sectional image, the average of the numbers of black pixels that are continuous in the Z axis direction is calculated in the same manner.
5) The averages of the numbers of black pixels that are continuous in the Z axis direction calculated in the respective lines are further averaged to calculate an average pore length in the Z axis direction. This is referred to as Coredlength in the Z axis direction.
6) An average pore length in the X axis direction (Coredlength in the X axis direction) is determined in the same manner as in the above 3) and 4) from horizontal (X axis direction) lines of 1 pixel width in the sectional image.
7) The obtained Coredlength in the Z axis direction is defined as a pore length (Lt) in a thickness direction perpendicular to the surface of the element body 101a. The obtained Coredlength in the X axis direction is defined as a pore length (Lf) in a surface direction perpendicular to the thickness direction. Using these values, the ratio (Lt/Lf) of the pore length (Lt) in the thickness direction to the pore length (Lf) in the surface direction is calculated.

Instead of the Coredlength in the X axis direction, Coredlength in the Y axis direction may be calculated using the image of the YZ section. The obtained Coredlength in the Y axis direction may be defined as a pore length (Lf) in a surface direction perpendicular to the thickness direction. This is because the Coredlength in the X axis direction and the Coredlength in the Y axis direction are considered to be equivalent.

The ratio (Lt/Lf) in each of the porous protective layers 91b to 91e can also be calculated in the same manner. However, in the porous protective layers 91c and 91d, the Coredlength in the X axis direction is defined as a pore length (Lt) in the thickness direction, and either the Coredlength in the Y axis direction or the Coredlength in the Z axis direction is defined as a pore length (Lf) in the surface direction. Further, in the porous protective layer 91e, the Coredlength in the Y axis direction is defined as a pore length (Lt) in the thickness direction, and either the Coredlength in the X axis direction or the Coredlength in the Z axis direction is defined as a pore length (Lf) in the surface direction.

In this embodiment, in all the porous protective layers 91a to 91e, the ratio (Lt/Lf) is regarded as the same value within the range of 0.6 to 0.9.

It is to be noted that the porous protective layer 91 is considered to have substantially the same microstructure regardless of observation area. Therefore, a ratio (Lt/Lf) determined using an arbitrary sectional image as described above may be used as the ratio (Lt/Lf) in the porous protective layer 91. For example, the ratio (Lt/Lf) in the porous protective layer 91a may be used as the ratio (Lt/Lf) in the porous protective layer 91.

As described above, the porous protective layer 91 has a structure having a pore in which the ratio (Lt/Lf) is 0.6 to 0.9. That is, pores in the porous protective layer 91 averagely have a flat shape that spreads in the surface direction to be thin in the thickness direction. Therefore, it is considered that a sufficient number of pores to achieve heat insulating properties are easily disposed in the thickness direction regardless of the position of surface direction of the porous protective layer 91. This effect is easily obtained when the ratio (Lt/Lf) in a pore is 0.9 or less. The upper limit of the ratio (Lt/Lf) in a pore may also be 0.85 or less or 0.8 or less. It is considered that when water is splashed on the surface of such a porous protective layer 91, its temperature change in the thickness direction can further be decreased, which further reduces thermal shock applied to the element body 101a. As a result, the water resistance of the sensor element 101 can be improved.

Further, it is considered that when the ratio (Lt/Lf) in a pore is 0.6 or more, the pore does not excessively spread in the surface direction, which makes it difficult for the porous protective layer 91 to be peeled off. As a result, the porous protective layer 91 can maintain necessary strength. From the viewpoint of peel resistance, the lower limit of the ratio (Lt/Lf) in a pore may also be 0.65 or more or 0.7 or more.

The thickness of the porous protective layer 91 may be, for example, 100 μm or more and 1000 μm or less. The thickness of the porous protective layer 91 may be 100 μm or more and 500 μm or less. The thickness is determined in the following manner using an image (SEM image) obtained by observation with a scanning electron microscope (SEM). In an area where the porous protective layer 91 is present, the sensor element 101 is cut orthogonally to the longitudinal direction of the sensor element 101. The cut surface is embedded in a resin and polished to prepare an observation sample. The magnification of the SEM is set to 80 times, and the surface to be observed of the observation sample is imaged to obtain an SEM image of section of the porous protective layer 91a. A direction perpendicular to the surface of the element body 101a is defined as a thickness direction, a distance between the surface of the porous protective layer 91a and the interface with the element body 101a is determined, and the distance is defined as the thickness of the porous protective layer 91a. It is to be noted that the porous protective layer 91a is formed as a layer having a predetermined thickness. Therefore, the thickness determined using one sectional image as described above may be used as the thickness of the porous protective layer 91a. The thickness of each of the porous protective layers 91b to 91e is also determined in the same manner.

In this embodiment, all the porous protective layers 91a to 91e have the same thickness. However, the porous protective layers 91a to 91e may be different from each other in thickness.

The porosity of the porous protective layer 91 may be, for example, 10% by volume to 70% by volume. Alternatively, the porosity may be 10% by volume to 40% by volume. The porosity is determined in the following manner using an image (SEM image) obtained by observation with a scanning electron microscope (SEM). As in the case of determination of the thickness described above, the magnification of the SEM is set to 80 times, and the SEM image of section of the porous protective layer 91a is obtained. Then, the obtained SEM image is binarized using “Otsu's method” (also referred to as discriminant analysis method)). In the binarized image, alumina is shown in white and pores are shown in black. In the binarized image, area of alumina portions (white) and area of pore portions (black) are obtained. The ratio of the area of the pore portions to total area (total of the area of the alumina portions and the area of the pore portions) is calculated and defined as porosity. The porosity of each of the porous protective layers 91b to 91e is also determined in the same manner. In this embodiment, all the porous protective layers 91a to 91e have the same porosity.

It is to be noted that the porous protective layer 91 is considered to have substantially the same microstructure regardless of observation area. Therefore, as described above, the porosity determined using one sectional image may be used as the porosity of the porous protective layer 91.

The porous protective layer 91 may be a single layer or may comprise two or more layers. That is, the porous protective layer 91 may comprise a surface layer and an inner layer formed inside the surface layer. The surface layer and the inner layer may be different in constituent material or porosity. The porosity of the inner layer is preferably higher than that of the surface layer. The porosity of the inner layer may be, for example, 40% by volume or more and 70% by volume or less. The porosity of the surface layer may be, for example, 10% by volume or more and 40% by volume or less.

The porous protective layer 91 may have two or more inner layers. It is preferred that the porosity of at least one inner layer is higher than that of the surface layer. The two or more inner layers may be formed so that the porosity increases from the surface layer toward the inside.

A thickness of the surface layer in the porous protective layer 91 may be 100 μm or more and 300 μm or less. A thickness of the inner layer in the protective layer 91 may be 300 μm or more and 700 μm or less. When two or more inner layers are formed in the protective layer 91, total thickness of the inner layers may be 300 μm or more and 700 μm or less.

Generally, the heat insulating performance of a porous material improves as the porosity of the porous material increases. However, the structural strength of the porous material improves as the porosity of the porous material decreases. When the porous protective layer 91 comprises a surface layer and an inner layer having a higher porosity than the surface layer, the structural strength of the porous protective layer 91 can be maintained by the surface layer while the heat insulating effect of the porous protective layer 91 can be improved by the inner layer having a high porosity. Therefore, the strength of the porous protective layer 91 can be maintained while the water resistance of the sensor element 101 can be improved. Further, an amount of a measurement-object gas flowing into the gas inlet 10 can also be adjusted by the diffusion resistance of the surface layer.

### [Production Method of Sensor Element]

Hereinbelow, an example of a method for producing such a sensor element as described above will be described. In the production method of the sensor element 101, the element body 101a is first produced, and then the porous protective layer 91 is formed on the element body 101a to produce the sensor element 101.

Hereinafter, description is made while taking the case of manufacturing the sensor element 101 composed of six layers shown in FIG. 2 as an example.

### (Production of Element Body)

First, a method for producing the element body 101a will be described. Six green sheets containing an oxygen-ion-conductive solid electrolyte such as zirconia (ZrO₂) as a ceramic component are prepared. For manufacturing of the green sheets, a known molding method can be used. The six green sheets may all have the same thickness, or the thickness differs depending on the layer to be formed. In each of the six green sheets, sheet holes or the like for use in positioning at the time of printing or stacking are formed in advance by a known method such as a punching process with a punching apparatus (blank sheet). In the blank sheet for use as the spacer layer 5, penetrating parts such as internal cavities are also formed in the same manner. Also in the remaining layers, necessary penetrating parts are formed in advance.

The blank sheets for use as six layers, namely, the first substrate layer 1, the second substrate layer 2, the third substrate layer 3, the first solid electrolyte layer 4, the spacer layer 5, and the second solid electrolyte layer 6 are subjected to printing of various patterns required for respective layers and drying treatment. For printing of a pattern, a known screen printing technique can be used. Also as the drying treatment, a known drying means can be used.

After completing the printing and drying of diverse patterns for each of the six blank sheets by repeating these steps, contact bonding treatment of stacking the six printed blank sheets in a predetermined order while positioning with the sheet holes and the like, and contact bonding at a predetermined temperature and pressure condition to give a laminate is conducted. The contact bonding treatment is conducted by heating and pressurizing with a known laminator such as a hydraulic press. While the temperature, the pressure and the time of heating and pressurizing depend on the laminator being used, they may be appropriately determined to achieve excellent lamination.

The obtained laminate includes a plurality of element bodies 101a. The laminate is cut into units of the element body 101a. The cut laminate is fired at a predetermined firing temperature to obtain the element body 101a. The firing temperature may be such a temperature that the solid electrolyte forming the base part 102 of the sensor element 101 is sintered to become a dense product, and an electrode or the like maintains desired porosity. The firing is conducted, for example, at a firing temperature of about 1300 to 1500° C.

### (Production of Protective Layer)

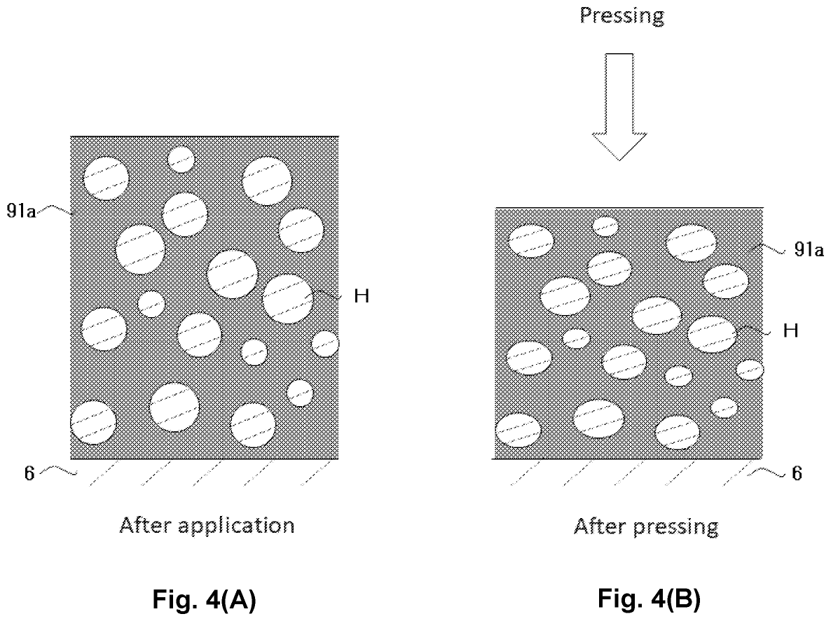

Next, a method for forming the porous protective layer 91 on the element body 101a will be described. In this embodiment, the porous protective layer 91 is formed through the steps of application, pressing, and degreasing. FIG. 4(A) is a schematic diagram simply showing the shape of pore precursors H in the section of the porous protective layer 91a after application, and FIG. 4(B) is a schematic diagram simply showing the shape of the pore precursors H in the section of the porous protective layer 91a after pressing. In the pore precursors H, a pore forming material is present. After the pressing step, the pore forming material in the pore precursors H disappears in the degreasing step so that pores are formed there. As a result, the porous protective layer 91a having such a sectional structure as schematically shown in FIG. 3(B) is obtained.

First, a protective layer forming composition including a pore forming material for use in the application step is prepared. In this embodiment, a porous protective layer paste is prepared as the protective layer forming composition. The porous protective layer paste is prepared by blending a raw material powder comprising the above-described material of the porous protective layer 91 (in this embodiment, an alumina powder), a pore forming material for forming pores, and an organic binder, an organic solvent, etc. The pore forming material is an organic or inorganic material that will disappear by degreasing in the subsequent step. Examples of the pore forming material that can be used include a xanthine derivative such as theobromine, an organic resin material such as an acrylic resin, and an inorganic material such as carbon. The porous protective layer paste is preferably prepared so that the porosity of the porous protective layer 91 after degreasing is 10% by volume to 40% by volume. The porosity of the porous protective layer 91 may be adjusted to fall within a desired range by, for example, adjusting the amount of the pore forming material to be added. For example, the porous protective layer 91 having a porosity of 10% by volume to 40% by volume may be obtained by adding the pore forming material at 10% by volume to 50% by volume relative to the alumina powder. Alternatively, the porosity of the porous protective layer 91 may be adjusted by adjusting the particle diameter of the raw material powder or the blending ratio of the organic binder.

Then, the step of forming a coating layer is performed by applying the protective layer forming composition including the pore forming material onto at least a part of the surface of the element body 101a. In this embodiment, application by screen printing is given as an example. The porous protective layer paste is printed and dried on a part of the top surface of the element body 101a where the porous protective layer 91a is to be formed to form a coating layer of the porous protective layer 91a. The printing can be performed using a known screen printing technique. The drying can also be performed using a known drying means. During the drying, the pore forming material does not evaporate and remains in the coating layer as pore precursors H. The printing and drying may repeatedly be performed.

The porous protective layers 91b to 91e are also formed by performing printing and drying in the same manner. The order of forming the porous protective layers 91a to 91e by printing and drying is not particularly limited.

A printed film thickness of the porous protective layer 91 may appropriately be determined by those skilled in the art, on the basis of a predetermined thickness (thickness after degreasing) of the porous protective layer 91 in the sensor element 101 in consideration of the degree of compression caused by pressing and shrinkage caused by degreasing in the subsequent steps.

Then, the step of pressing the coating layer is performed. The coating layer of the porous protective layer 91 is compressed by pressing so that the ratio (Lt/Lf) in the porous protective layer 91 after degreasing is 0.6 to 0.9. The degree of pressing (the degree of compression of coating layer of the porous protective layer 91) can appropriately be set by those skilled in the art on the basis of a predetermined ratio (Lt/Lf) in the porous protective layer 91 after degreasing.

The pressing may be performed three times by separately pressing the top and bottom surfaces, the right and left surfaces, and the front end surface using a uniaxial pressing device such as a known hydraulic pressing machine. Alternatively, a cold isostatic pressing (CIP) device or the like may be used. The pressure, temperature, and time during the pressing depend on the pressing device to be used, but may appropriately be set so that a desired degree of pressing is achieved.

Finally, the step of subjecting the coating layer to heat treatment is performed to obtain the porous protective layer 91 comprising a porous material. That is, the step of degreasing is performed at a predetermined degreasing temperature. The degreasing temperature is not particularly limited as long as all the pore forming material, the organic binder, the organic solvent, etc. contained as organic components in the printed film of the porous protective layer 91 can disappear and the porous structure of the porous protective layer 91 can be maintained. The degreasing temperature may be lower than the firing temperature of the element body 101a. For example, the coating layer is degreased at a degreasing temperature of about 400 to 900° C.

In the above-described production method, the layer to be served as the porous protective layer 91 is formed by application using screen printing and then pressing and degreasing. However, the method for producing the porous protective layer according to the present invention is not limited thereto. Depending on the degree of shrinkage during heat treatment such as degreasing or firing, there is a case where the sensor element according to the present invention can be produced without pressing.

Alternatively, the layer may be applied by dipping and then be pressed. Further, the layer may be formed by, for example, plasma spraying or gel casting and then be pressed.

There is also a case where the sensor element according to the present invention can be produced by optimizing the spray conditions of plasma spraying.

The obtained sensor element 101 is incorporated into the gas sensor 100 in such a form that the front end part of the sensor element 101 comes into contact with the measurement-object gas, and the rear end part of the sensor element 101 comes into contact with the reference gas.

In the above-described embodiment, the element body 101a has a flat surface and an almost rectangular section. However, the element body according to the present invention is not limited thereto. The element body 101a may have a curved surface. Further, the element body 101a may have an almost circular or elliptical section (for example, a bottomed cylindrical oxygen sensor element such as one disclosed in Japanese Patent No. 3766572). Each of the components of the element body may also be variously embodied.

### EXAMPLES

Hereinafter, the case of actually manufacturing a sensor element and conducting a test is described as Examples. Experimental Examples 2 to 4 correspond to Examples of the present invention, and Experimental Examples 1 and 5 correspond to Comparative Examples of the present invention. The present invention is not limited to the following Examples.

### Experimental Examples 1 to 4

As Experimental Examples 1 to 4, a sensor element 101 having a porous protective layer 91 in which the ratio (Lt/Lf) was 0.5 (Experimental Example 1), a sensor element 101 having a porous protective layer 91 in which the ratio (Lt/Lf) was 0.7 (Experimental Example 2), a sensor element 101 having a porous protective layer 91 in which the ratio (Lt/Lf) was 0.8 (Experimental Example 3), and a sensor element 101 having a porous protective layer 91 in which the ratio (Lt/Lf) was 0.9 (Experimental Example 4) were produced in accordance with the above-described production method of a sensor element 101. In each of Experimental Examples 1 to 4, the porous protective layer 91 had a thickness of 300 μm and a porosity of 30% by volume.

Specifically, an element body 101a was produced which had a longitudinal length of 67.5 mm, a horizontal width of 4.25 mm, and a vertical thickness of 1.45 mm.

A porous protective layer paste was prepared by blending an alumina powder with a pore forming material in a ratio of 30% by volume based on the alumina powder and adding a solvent, a binder, and a dispersant thereto.

Then, a porous protective layer 91 was formed on the surface of the element body 101a. The porous protective layer paste was applied by screen printing, and then the pressing step was performed. The degreasing step was performed to produce the sensor elements 101 of Experimental Examples 1 to 4. In the pressing step, the degree of pressing was changed to allow the Experimental Examples 1 to 4 to achieve their respective desired ratios (Lt/Lf).

In the pressing step, a hot press was used as a pressing device.

In each of Experimental Examples 1 to 4, a printed film thickness and an applied pressure were adjusted so that the porous protective layer 91 had a thickness of 300 μm and a desired ratio (Lt/Lf). The ratio (Lt/Lf) was decreased by increasing the printed film thickness and the applied pressure.

The degreasing temperature was set to 600° C.

### Experimental Example 5

As Experimental Example 5, a sensor element 101 having a porous protective layer 91 in which the ratio (Lt/Lf) was 1 was produced. As in Experimental Examples 1 to 4, the porous protective layer 91 of Experimental Example 5 had a thickness of 300 μm and a porosity of 30% by volume. The sensor element 101 was produced in the same manner as in Experimental Examples 1 to 4 except that the pressing step was not performed.

[Confirmation of ratio (Lt/Lf)]

The porous protective layers 91 of the sensor elements 101 of Experimental Examples 1 to 5 were imaged by CT (Versa520 manufactured by Carl Zeiss, 140 kV, 10 W). Using the above-described technique, Experimental Examples 1 to 5 were confirmed to achieve their respective desired ratios (Lt/Lf).

### [Evaluation of Water Resistance]

The sensor elements 101 of Experimental Examples 1 to 5 were subjected to evaluation of the performance of the porous protective layer 91 (water resistance of the sensor element 101). Specifically, initially, the heater 72 was energized, the temperature was set at 800° C., and the sensor element 101 was heated. In this state, the main pump cell 21, the auxiliary pump cell 50, the oxygen-partial-pressure detection sensor cell 80 for main pump control, the oxygen-partial-pressure detection sensor cell 81 for auxiliary pump control, and the like were actuated in an air atmosphere and the oxygen concentration in the first internal cavity 20 was controlled so as to be maintained at a predetermined constant value. Then, after waiting stabilization of the pump current Ip0, water was dropped on the top surface of the porous protective layer 91 (the porous protective layer 91a), and presence or absence of a crack in the sensor element 101 was determined on the basis of whether the pump current Ip0 changed to a value exceeding a predetermined threshold value or not. In this regard, if cracking occurs in the sensor element 101 because of thermal shock due to a water droplet, oxygen passes through the cracked portion and flows into the first internal cavity 20 easily, so that the value of the pump current Ip0 increases. Therefore, in the case where the pump current Ip0 exceeded the predetermined threshold value determined on the basis of an experiment, it was judged that cracking occurred in the sensor element 101 because of the water droplet. A plurality of tests were performed while the amount of the water droplet was increased up to 30 μL gradually, and the maximum amount of the water droplet, at which cracking did not occur, was taken as an amount indicating the water resistance. Then, each five sensor elements 101 of each of Experimental examples 1 to 5 were prepared, and an average value of amounts indicating the water resistance of the five sensor elements 101 was derived in each of Experimental examples 1 to 5. The water resistance of the sensor element 101 of each of Experimental examples 1 to 5 was evaluated, where the average value of the amount indicating the water resistance of less than 10 μL was specified to be no good, 10 μL or more was specified to be good.

### [Evaluation of Peeling Resistance]

The sensor elements 101 of Experimental Examples 1 to 5 were subjected to evaluation of the peeling resistance of the porous protective layer 91. Specifically, gas sensors 100 of Experimental Examples 1 to 5 respectively including the sensor elements 101 of Experimental Examples 1 to 5 were produced. The number of gas sensors 100 produced in each of Experimental Examples 1 to 5 was 5. A hot vibration test was performed under the following conditions in a state where each of the gas sensors 100 of Experimental Examples 1 to 5 was attached to the exhaust pipe of a propane burner placed in a vibration tester.

Gas temperature: 850° C.;

Gas air ratio λ:1.05;

Vibration conditions: sweeping for 30 minutes at 50 Hz, 100 Hz, 150 Hz, and 250 Hz in this order;

Acceleration: 30 G, 40 G, and 50 G; and

Test time: 150 hours.

The sensor element 101 was taken out of each of the gas sensors 100 of Experimental Examples 1 to 5 after the hot vibration test. The porous protective layer 91 of each of the five sensor elements 101 of each of Experimental Examples 1 to 5 after the hot vibration test was observed using a scanning electron microscope (SEM). Specifically, in an area where the porous protective layer 91 was present, the sensor element 101 was cut orthogonally to the longitudinal direction of the sensor element 101. The cut section was embedded in a resin and polished and was then observed with a SEM at magnifications of 80 times and 500 times to determine whether the porous protective layer 91 was peeled off or not. The peeling resistance of the sensor elements 101 of Experimental Examples 1 to 5 was evaluated. The peeling resistance was evaluated as good when peeling-off was not observed and no good when peeling-off was observed.

The ratios (Lt/Lf) and the evaluation results of water resistance and peeling resistance of the sensor elements 101 of Experimental Examples 1 to 5 are shown in Table 1.

TABLE 1

| | Ratio | Water | Peeling |
|---|---|---|---|
| | (Lt/Lf) | resistance | resistance |
| Experimental | 0.5 | Good | No Good |
| Example 1 | | | |
| (Comparative) | | | |
| Experimental | 0.7 | Good | Good |
| Example 2 | | | |
| Experimental | 0.8 | Good | Good |
| Example 3 | | | |
| Experimental | 0.9 | Good | Good |
| Example 4 | | | |
| Experimental | 1 | No Good | Good |
| Example 5 | | | |
| (Comparative) | | | |

As shown in Table 1, it was confirmed that excellent water resistance was achieved when the ratio (Lt/Lf) in the porous protective layer 91 was 0.9 or less. It was also confirmed that excellent peeling resistance was achieved when the ratio (Lt/Lf) was 0.6 or more.

### EXPLANATION OF REFERENCE SIGNS IN THE DRAWINGS

1: first substrate layer; 2: second substrate layer; 3: third substrate layer; 4: first solid electrolyte layer; 5: spacer layer; 6: second solid electrolyte layer; 10: gas inlet; 11: first diffusion-rate limiting part; 12: buffer space; 13: second diffusion-rate limiting part; 15: measurement-object gas flow part; 20: first internal cavity; 21: main pump cell; 22: inner main pump electrode; 22a: ceiling electrode portion (of the inner main pump electrode); 22b: bottom electrode portion (of the inner main pump electrode); 23: outer pump electrode; 24: variable power supply (of the main pump cell); 30: third diffusion-rate limiting part; 40: second internal cavity; 41: measurement pump cell; 42: reference electrode; 43: reference gas introduction space; 44: measurement electrode; 46: variable power supply (of the measurement pump cell); 48: air introduction layer; 50: auxiliary pump cell; 51: auxiliary pump electrode; 51a: ceiling electrode portion (of the auxiliary pump electrode); 51b: bottom electrode portion (of the auxiliary pump electrode); 52: variable power supply (of the auxiliary pump cell); 60: fourth diffusion-rate limiting part; 61: third internal cavity; 70: heater part; 71: heater electrode; 72: heater; 73: through hole; 74: heater insulating layer; 75: pressure relief vent; 76: heater lead; 80: oxygen-partial-pressure detection sensor cell for main pump control; 81: oxygen-partial-pressure detection sensor cell for auxiliary pump control; 82: oxygen-partial-pressure detection sensor cell for measurement pump control; 83: sensor cell; 91, 91a to 91e: porous protective layer; 100: gas sensor; 101: sensor element; 101a: element body; and 102: base part.

What is claimed is:

1. A sensor element for detecting a target gas to be measured in a measurement-object gas, the sensor element comprising:
an element body including an oxygen-ion-conductive solid electrolyte layer; and
a protective layer covering at least a part of a surface of the element body, wherein
the protective layer comprises a porous material that has a pore inside; and
in the pore in the protective layer, a ratio (Lt/Lf) of a pore length (Lt) in a thickness direction perpendicular to the surface of the element body to a pore length (Lf) in a surface direction perpendicular to said thickness direction is 0.6 to 0.9.

2. The sensor element according to claim 1, wherein the protective layer has a thickness of 100 μm to 500 μm.

3. The sensor element according to claim 1, wherein the protective layer has a porosity of 10% by volume to 40% by volume.

4. The sensor element according to claim 1, wherein the protective layer comprises a surface layer, and an inner layer formed inside the surface layer; and
the inner layer has a higher porosity than the surface layer.

5. The sensor element according to claim 4, wherein the inner layer in the protective layer has a thickness of 300 μm to 700 μm.

6. The sensor element according to claim 4, wherein the surface layer in the protective layer has a thickness of 100 μm to 300 μm.

7. The sensor element according to claim 4, wherein the inner layer in the protective layer has a porosity of 40% by volume to 70% by volume.

8. The sensor element according to claim 1, wherein the sensor body comprises:
a base part in an elongated plate shape, including a plurality of oxygen-ion-conductive solid electrolyte layers stacked;
a measurement-object gas flow part formed from one end part in a longitudinal direction of the base part;
at least one inner electrode disposed on an inner surface of the measurement-object gas flow part; and
an outer electrode disposed in contact with the inner electrode via at least one layer of the plurality of oxygen-ion-conductive solid electrolyte layers.

9. A production method of a sensor element according to claim 1, the production method comprising the steps of:
applying a protective layer forming composition including a pore forming material, onto at least a part of a surface of the element body, to form a coating layer;
pressing the coating layer; and
degreasing the pressed coating layer to obtain a protective layer comprising a porous material.
